(19) United States
(12) Patent Application Publication
(10) Pub. No.: US 2006/0223999 A1
(43) Pub. Date: Oct. 5, 2006

(54) Process for preparing montelukast and precursors thereof

(75) Inventors: Jingshan Shen, Shanghai (CN); Yiru Dai, Shanghai (CN); Joseph Kaspi, Givatayim (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: CHEMAGIS LTD., Bnei Brak (IL)

(21) Appl. No.: 11/431,730

(22) Filed: May 10, 2006

(51) Int. Cl. C07D 215/14; C07D 215/12

(52) U.S. Cl. 546/174

(57) ABSTRACT

The present invention provides a process for stereoselectively reducing 2-[3-[3-[2-(7-chloro-2-quinolinyl)ethenyl]-phenyl]-3-oxopropyyl]benzoic-acid methyl ester, to produce to produce methyl 2-[3-(S)-[3-[2-(7-chloro-2-quinolinyl)-ethenyl]phenyl]-3-hydroxypropyl]benzoate, and a process for producing montelukast or a salt thereof. The present invention further provides a process for purifying methyl 2-[3-(S)-[3-[2-(7-chloro-2-quinolinyl)-ethenyl]phenyl]-3-hydroxypropyl]benzoate. The reduction process of the present invention uses a chiral reagent and can produce the desired reduction product in high enantiomeric excess (ee).

### BACKGROUND OF THE INVENTION

(R-(E))-1-(((1-(3-(2-(7-chloro-2-quinolinyl)ethenyl)phenyl)-3-(2-(1-hydroxy-1-methylethyl)phenyl)propyl)thio)methyl)cyclopropaneacetic acid sodium salt, also known by the name of montelukast sodium, is represented by the structural formula 1 below:

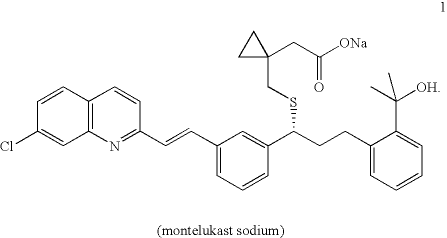

Montelukast sodium is a leukotriene antagonist, and is thus useful as an anti-asthmatic, anti-allergic, anti-inflammatory and cytoprotective agent. Montelukast sodium is currently indicated for the treatment of allergic rhinitis and asthma.

Montelukast sodium and related compounds were first disclosed in EP 0 480 717. The process for preparing montelukast sodium according to EP 0 480 717 comprises stereoselective reduction of the intermediate 2-[3-[3-[2-(7-chloro-2-quinolinyl)-ethenyl]phenyl]-3-oxopropyl]benzoic acid methyl ester 2 using (−)-B-chlorodiisopinocamphenylborane 3 or the S-oxazaborolidine 4 to obtain methyl 2-[3-(S)-[3-[2-(7-chloro-2-quinolinyl)-ethenyl]phenyl]-3-hydroxypropyl]benzoate 5.

The process for preparing the montelukast precursor is depicted in Scheme 1 below.

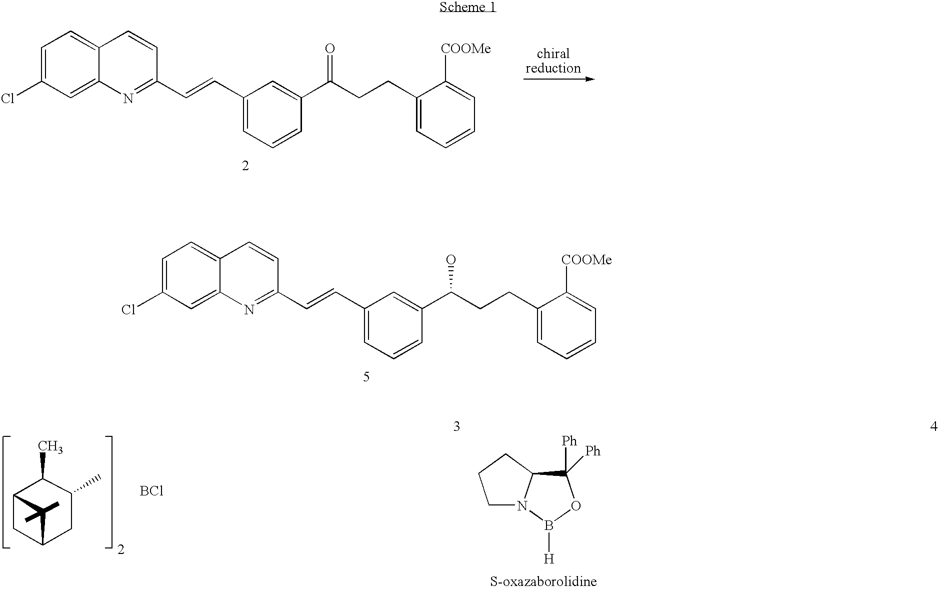

U.S. Pat. Nos. 4,772,752 and 5,043,479 describe the preparation and use of mono and diisopinocamphenylhaloboranes as chiral reducing agents, U.S. Pat. No. 5,292,946 describes an in-situ preparation of diisopinocamphenylchloroborane and the use thereof in reduction of prochiral ketones to optically active alcohols, and U.S. Pat. Nos. 5,545,758 and 5,693,816 describe processes for the preparation of diisopinocamphenylchloroborane and the use thereof for reduction of the intermediate 2-[3-[3-[2-(7-chloro-2-quinolinyl)-ethenyl]phenyl]-3-oxopropyl]benzoic acid methyl ester 2 to methyl 2-[3-(S)-[3-[2-(7-chloro-2-quinolinyl)-ethenyl]phenyl]-3-hydroxypropyl]benzoate 5.

U.S. Pat. No. 6,184,381 describes a process for stereoselective reduction of the intermediate 2-[3-[3-[2-(7-chloro-2-quinolinyl)-ethenyl]phenyl]-3-oxopropyl]benzoic acid methyl ester 2 using an optically active ruthenium-diamine complex to obtain compound 5, and WO 2006/008562 describes the use of a chiral ruthenium or rhodium catalyst, in the presence of a hydrogen source. However, the catalysts are not commercially available and have to be specially prepared, hence the process is more complicated and expensive.

The stereoselective reduction step of compound 2, as described in EP 0 480 717, uses the reagent (−)-B-chlorodiisopinocamphenylborane, which is an expensive and unstable reagent, and the reaction is carried out at a temperature of −25° C., which is inconvenient for large-scale industrial implementation. Furthermore, according to example 146 step 2 of patent EP 0 480 717, the molar ratio between the reagent (−)-B-chlorodiisopinocamphenylborane and the ketone 2 is high namely 1.5:1.

The foregoing processes for stereoselectively reducing compound 2 to compound 5 suffer from high catalyst ratio, low reaction temperatures, expensive or unstable catalysts, or usage of catalysts that are not commercially available. There is a need in the art for an improved reduction process useful in the production of montelukast and salts thereof using reagents, which are not expensive and readily available. The present invention provides such a process.

### SUMMARY OF THE INVENTION

The present invention provides a process for producing montelukast or a salt thereof, which process includes stereoselectively reducing 2-[3-[3-[2-(7-chloro-2-quinolinyl)ethenyl]-phenyl]-3-oxopropyl]benzoic acid methyl ester 2 in the presence of a catalytic amount of a chiral reduction catalyst, which is preferably (R)-methyl oxazaborolidine (MeCBS) 6, (−)-B-bromodiisopinocamphenylborane, trans-RuH(η¹BH₄)[(R)-2,2′-bis(di-4-tolylphosphino)-1,1′-binaphthyl][(R,R)-1,2-diphenyl-ethylenediamine, trans-RuCl₂[(R)-2,2′-bis(di-3,5-dimethylphenylphosphino)]-1,1′-binaphthyl][(R,R)-1,2-diphenylethylenediamine], or [[N(S), N′(S), 1R, 2R]-N,N′-bis-[[2-(diphenylphosphino)-phenyl]methyl]1,2-cyclohexanediamine-N,N′,P,P′]-dichloro-ruthenium, to produce methyl 2-[3-(S)-[3-[2-(7-chloro-2-quinolinyl)-ethenyl]phenyl]-3-hydroxypropyl]benzoate 5; and converting the methyl 2-[3-(S)-[3-[2-(7-chloro-2-quinolinyl)-ethenyl]phenyl]-3-hydroxypropyl]benzoate 5 into montelukast or a salt thereof.

The present invention additionally provides a process for producing methyl 2-[3-(S)-[3-[2-(7-chloro-2-quinolinyl)-ethenyl]phenyl]-3-hydroxypropyl]benzoate 5, which process includes stereoselectively reducing 2-[3-[3-[2-(7-chloro-2-quinolinyl)ethenyl]-phenyl]-3-oxopropyl]benzoic acid methyl ester 2 in the presence of a catalytic amount of a chiral reduction catalyst, which preferably includes (R)-methyl oxazaborolidine (MeCBS) 6, (−)-B-bromodiisopinocamphenylborane, trans-RuH(η¹BH₄)[(R)-2,2′-bis(di-4-tolylphosphino)-1,1′-binaphthyl][(R,R)-1,2-diphenyl-ethylenediamine, trans-RuCl₂[(R)-2,2′-bis(di-3,5-dimethylphenylphosphino)]-1,1′-binaphthyl][(R,R)-1,2-diphenylethylenediamine], or [[N(S), N′(S),1R, 2R]-N,N′-bis-[[2-(diphenylphosphino)-phenyl]methyl]1,2-cyclohexanediamine-N,N′,P,P′]-dichloro-ruthenium. A preferred chiral reduction catalyst is (R)-methyl oxazaborolidine (MeCBS) 6.

The present invention further provides a process for purifying crude methyl 2-[3-(S)-[3-[2-(7-chloro-2-quinolinyl)-ethenyl]phenyl]-3-hydroxypropyl]benzoate 5, which process preferably includes dissolving the crude compound 5 in a polar organic solvent; adding a non-polar solvent and water; optionally adding another portion of the non-polar solvent cooling for sufficient time period to produce crystals of compound 5; optionally isolating the product; and, optionally washing and drying the crystals.

### DETAILED DESCRIPTION OF THE INVENTION

The present invention provides a process for producing montelukast or a salt thereof, which process includes stereoselectively reducing 2-[3-[3-[2-(7-chloro-2-quinolinyl)ethenyl]-phenyl]-3-oxopropyl]benzoic acid methyl ester 2 in the presence of a catalytic amount of a chiral reduction catalyst, which is preferably (R)-methyl oxazaborolidine (MeCBS) 6, (−)-B-bromodiisopinocamphenylborane, trans-RuH(η¹BH₄) [(R)-2,2′-bis(di-4-tolylphosphino)-1,1′-binaphthyl][(R,R)-1,2-diphePnyl-ethylenediamrine, trans-RuCl₂[(R)-2,2′-bis(di-3,5-dimethylphenylphosphino)]-1,1′-binaphthyl][(R,R)-1,2-diphenylethylenediamine], or [[N(S),N′(S), 1R, 2R]-N,N′-bis-[[2-(diphenylphosphino)-phenyl]methyl]1,2-cyclohexanediamine-N,N′,P,P′]-dichloro-ruthenium, to produce methyl 2-[3-(S)-[3-[2-(7-chloro-2-quinolinyl)-ethenyl]phenyl]-3-hydroxypropyl]benzoate 5; and converting the methyl 2-[3-(S)-[3-[2-(7-chloro-2-quinolinyl)-ethenyl]phenyl]-3-hydroxypropyl]benzoate 5 into montelukast or a salt thereof. Processes for converting methyl 2-[3-(S)-[3-[2-(7-chloro-2-quinolinyl)-ethenyl]phenyl]-3-hydroxypropyl]benzoate 5 into montelukast or a salt thereof (e.g., montelukast sodium) are well known in the art. Suitable processes for converting methyl 2-[3-(S)-[3-[2-(7-chloro-2-quinolinyl)-ethenyl]phenyl]-3-hydroxypropyl]benzoate 5 into montelukast or a salt thereof are described, for example, in EP 0 480 717.

The present invention additionally provides a process for producing methyl 2-[3-(S)-[3-[2-(7-chloro-2-quinolinyl)-ethenyl]phenyl]-3-hydroxypropyl]benzoate 5, which process includes stereoselectively reducing 2-[3-[3-[2-(7-chloro-2-quinolinyl)ethenyl]-phenyl]-3-oxopropyl]benzoic acid methyl ester 2 in the presence of a catalytic amount of a chiral reduction catalyst, which preferably includes (R)-methyl oxazaborolidine (MeCBS) 6, (−)-B-bromodiisopinocamphenylborane, trans-RuH(η¹BH₄)[(R)-2,2′-bis(di-4-tolylphosphino)-1,1′-binaphthyl][(R,R)-1,2-diphenyl-ethylenediamine, trans-RuCl₂[(R)-2,2′-bis(di-3,5-dimethylphenylphosphino)]-1,1′-binaphthyl][(R,R)-1,2-diphenylethylenediamine], or [[N(S),N′(S),1R, 2R]-N,N′-bis-[[2-(diphenylphosphino)-phenyl]methyl]1,2-cyclohexanediamine-N,N′,P,P′]-dichloro-ruthenium.

A preferred chiral reduction catalyst used in the process of the present invention includes (R)-methyl oxazaborolidine (hereinafter “MeCBS”) 6, which has the following molecular structure. 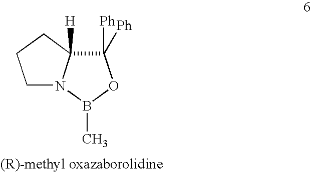
In this regard, the Applicants have surprisingly discovered that (R)-methyl oxazaborolidine (MeCBS) 6 is a suitable catalyst for performing the desired stereoselective reduction, and yet has the opposite stereochemical configuration of oxazaborolidine 4, which has a similar empirical structure (but is not identical to 6). Moreover, (R)-methyl oxazaborolidine (MeCBS) 6 is commercially available in large quantities (e.g., the BASF Corporation offers the reagent in cylinders of 90 liters).

In a preferred embodiment, the present invention provides a process for stereoselectively reducing the montelukast intermediate 2-[3-[3-[2-(7-chloro-2-quinolinyl)ethenyl]-phenyl]-3-oxopropyl]benzoic acid methyl ester 2, wherein the process includes: reducing 2-[3-[3-[2-(7-chloro-2-quinolinyl)ethenyl]-phenyl]-3-oxopropyl]benzoic acid methyl ester 2 in the presence of a chiral reduction catalyst (e.g., by reducing with borane in the presence of a catalytic amount of a chiral reduction catalyst) in an organic solvent; quenching the reaction mixture; separating the product from the reaction mixture; and optionally purifying the product. Preferably, the chiral reduction catalyst includes (R)-methyl oxazaborolidine (MeCBS), (−)-B-bromodiisopinocamphenylborane, trans-RuH(η¹BH₄) [(R)-2,2′-bis(di-4-tolylphosphino)-1,1′-binaphthyl][1,2-diphenyl-ethylenediamine, trans-RuCl₂[(R)-2,2′-bis(di-3,5-dimethylphenylphosphino)-1,1′-binaphthyl][1,2-diphenylethylenediamine], or [[N(S), N′(S), 1R, 2R]-N,N′-bis-[[2-(diphenylphosphino)phenyl]methyl]1,2-cyclohexanediamine-N,N′,P,P′]-dichloro-ruthenium. A particularly preferred chiral reduction catalyst is (R)-methyl oxazaborolidine 6.

Any suitable amount of chiral reduction catalyst can be used in the stereoselective reduction process of the present invention. According to one embodiment of the present invention, the reduction is performed in the presence of MeCBS, wherein the molar ratio between the MeCBS and the ketone 2 (MeCBS:ketone 2) is at least about 0.01:1, and more preferably is at least about 0.15:1 (e.g., 0.15:1). In this regard, the process of the present invention requires a much lower molar ratio of catalyst than the process described in EP 0 480 717, example 146, step 2, which describes reducing ketone 2 in the presence of (−)-B-chlorodiisopinocamphenylborane in a ratio of 1.5:1 (catalyst:ketone 2).

The reduction process can be carried out in any suitable solvent. Suitable solvents can include organic solvents, preferably tetrahydrofuran (THF), 2-methyl-tetrahydrofuran, diethyl ether, diisopropyl ether, t-butyl methyl ether, dichloromethane, ethyl acetate and mixtures thereof. A particularly preferred organic solvent, which can be used in the reduction process of the present invention is tetrahydrofuran (THF).

The reduction process of the present invention can be monitored using any suitable method for monitoring chemical reaction. In one embodiment, after the starting material (ketone 2) has been consumed (as monitored by TLC or by other methods), the reaction mixture is stirred for additional 2 hours at 10° C. in order to ensure completion of the reaction.

In some embodiments of the present invention, the reduction process of the present invention can be quenched by adding a suitable reagent such as, for example, by quenching with one or more protic solvents, e.g., one or more alcohols. In one embodiment, the reduction reaction is quenched by adding an alcohol, which is preferably methanol, ethanol, n-propanol, isopropanol, n-butanol, sec-butanol, isobutanol, or a mixture thereof, preferably methanol.

In some embodiments of the present invention, the reduction reaction mixture can be acidified, e.g., after quenching, to a suitable pH, e.g., a pH value in the range of 4-5, to form an acid addition salt of the catalyst, and to enable facile work-up procedure for obtaining the final product. For instance, the reduction reaction can be acidified using e.g., an organic acid or an inorganic acid such as, e.g., hydrochloric acid, hydrobromic acid, sulfuric acid, p-toluenesulfonic acid, trifluoroacetic acid and the like, and mixtures thereof. A preferred acid, which can be used for acidifying the reduction reaction mixture, is hydrochloric acid.

In some embodiments of the present invention, it may be desirable to add ketone 2 to the chiral reduction catalyst (e.g., by adding ketone 2 to a mixture containing a reducing agent and the chiral reduction catalyst) gradually over time. In one embodiment of the present invention, the ketone 2 is added drop-wise to MeCBS over a period of about 30 minutes, which is a significantly faster addition rate than the addition rate of about 2 hours used in most of the asymmetric reductions with MeCBS reported in the literature.

The reduction process of the present invention can be performed at any suitable temperature. When the chiral reduction catalyst is MeCBS, the reduction process is preferably performed at a temperature of about 10° C. Although the reaction rate is faster at a reaction temperature in the range of 20-25° C., it has been found that the yields can be very low when the reduction process is carried out in this temperature range.

The present invention further provides a process for purifying crude methyl 2-[3-(S)-[3-[2-(7-chloro-2-quinolinyl)-ethenyl]phenyl]-3-hydroxypropyl]benzoate 5, which process preferably includes: dissolving the crude compound 5 in a polar organic solvent; adding a non-polar solvent and water; optionally adding another portion of the non-polar solvent; cooling for sufficient time period to enable crystallization; optionally isolating the product, e.g., by filtration, and, optionally washing and drying the product. Exemplary polar solvents, which can be used in the purification process of the present invention include, acetonitrile, acetone, tetrahydrofuran (THF), 2-methyl-tetrahydrofaran, dichloromethane, ethyl acetate and mixtures thereof, preferably tetrahydrofuran (THF). Exemplary non-polar solvents, which can be used in the purification process of the present invention include, pentane, hexane, cyclohexane, heptane, petrol ether and mixtures thereof, preferably heptane. In one embodiment, the ratio between the non-polar solvent, the polar solvent and the water in the crystallization solvent mixture (non-polar solvent/polar solvent/water ratio) is about 12/5/0.125 v/v/v. If desired, the crystals can be isolated (e.g., by filtration) and washed with a suitable solvent (e.g., a suitable organic solvent or a suitable mixture of two or more organic solvents). Preferably, the crystals are washed with a mixture of THF and hexane in a ratio of about 1:6 (THF:hepane, v/v/v).

The stereoselective reduction process of the present invention preferably produces methyl 2-[3-(S)-[3-[2-(7-chloro-2-quinolinyl)-ethenyl]phenyl]-3-hydroxypropyl]benzoate 5 having an enantiomeric excess (ee) of at least about 98%, and more preferably having enantiomeric excess which is greater than or equal to about 99.6% (e.g., 99.6% ee or greater).

The following example further illustrates the invention but, of course, should not be construed as in any way limiting its scope.

### EXAMPLE 1

This example illustrates the reduction of 2-[3-[3-[2-(7-chloro-2-quinolinyl)ethenyl]-phenyl]-3-oxopropyl]benzoic acid methyl ester using the catalyst R—Me—CBS.

A dry 100 ml 3-necked flask equipped with an addition funnel, a nitrogen inlet and a magnetic stirrer, which was covered with aluminium sheet (so as to perform the reaction in the dark), was charged with BH₃-THF complex (1.0M solution in THF, 4.0 mmol) followed by addition of (R)-methyl oxazaborolidine 6 (0.6 mmol, 1.0M solution in toluene). After stirring the reaction mixture for 45 minutes at 10° C., a solution of 2-[3-[3-[2-(7-chloro-2-quinolinyl)ethenyl]-phenyl]-3-oxopropyl]benzoic acid methyl ester (1.82 g, 4.0 mmol, 98%) in dry THF (20 mL) was added drop-wise at a period of 30 minutes while maintaining the reaction temperature at 10° C. After completing the addition, the reaction mixture was stirred for 2 hours at 10° C. Finally, the reaction mixture was cooled to 0-5° C. and quenched by addition of 8 mL of methanol drop-wise. The cold bath was removed and the reaction was stirred until hydrogen evolution ceased. The resulting solution was poured into a 100 ml, round-bottomed flask and the reaction vessel was rinsed with 5 ml of methanol. The solvent was distilled under reduced pressure. An additional 10 ml of fresh methanol was added and the solvent was again distilled under reduced pressure. The residue was cooled to room temperature to afford an oil and then quenched by addition of 1.0M HCl (5 mL). Dichloromethane (3 5 mL) was added and the organic phase was washed first with brine (3 10 mL ), then with saturated NaHCO₃ (3 10 mL ), and again with brine (3 10 mL ). The layers were separated and the organic phase was filtered. The filtrate was concentrated to give an oil, which was dissolved in THF (5 ml). Heptane (5 ml) was added followed by addition of water (about 0.125 ml) to induce crystallization. Heptane was again added (7 ml) and the mixture was maintained at 0-5° C. for about 1 hour to complete crystallization. The crystals were obtained by filtration and the thus formed cake was washed with a mixture of ⅙ THF/heptane, until the filtrate became almost colorless, and the cake was dried at 40° C. under vacuum to obtain 1.27 g of compound 3 in 68.8% yield, having 86.5% purity and enantiomeric excess (ee) of 99.7%.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for producing montelukast or a salt thereof, the process comprising:
stereoselectively reducing 2-[3-[3-[2-(7-chloro-2-quinolinyl)ethenyl]-phenyl]-3-oxopropyl]benzoic acid methyl ester 2 in the presence of a catalytic amount of a chiral reduction catalyst comprising (R)-methyl oxazaborolidine (MeCBS) 6, (−)-B-bromodiisopinocamphenylborane, trans-RuH(η¹BH₄)[(R)-2,2′-bis(di-4-tolylphosphino)-1,1′-binaphthyl][(R,R)-1,2-diphenyl-ethylenediamine, trans-RuCl₂[(R)-2,2′-bis(di-3,5-dimethylphenylphosphino)]-1,1′-binaphthyl][(R,R)-1,2-diphenylethylenediamine], or [[N(S),N′(S), 1R, 2R]-N,N′-bis-[[2-(diphenylphosphino)-phenyl]methyl]1,2-cyclohexanediamine-N,N′,P,P′]-dichloro-ruthenium, to produce methyl 2-[3-(S)-[3-[2-(7-chloro-2-quinolinyl)-ethenyl]phenyl]-3-hydroxypropyl]benzoate 5; and
converting the methyl 2-[3-(S)-[3-[2-(7-chloro-2-quinolinyl)-ethenyl]phenyl]-3-hydroxypropyl]benzoate 5 into montelukast or a salt thereof.

2. The process of claim 1, wherein the catalyst is (R)-methyl oxazaborolidine (MeCBS) 6.

3. The process of claim 1, comprising:
reducing 2-[3-[3-[2-(7-chloro-2-quinolinyl)ethenyl]-phenyl]-3-oxopropyl]benzoic acid methyl ester 2 in the presence of the catalyst in an organic solvent;
quenching the reaction mixture;
separating the methyl 2-[3-(S)-[3-[2-(7-chloro-2-quinolinyl)-ethenyl]phenyl]-3-hydroxypropyl]benzoate 5 from the reaction mixture; and
optionally purifying the methyl 2-[3-(S)-[3-[2-(7-chloro-2-quinolinyl)-ethenyl]phenyl]-3-hydroxypropyl]benzoate 5.

4. The process of claim 3, wherein the catalyst is (R)-methyl oxazaborolidine (MeCBS) 6.

5. The process of claim 2, wherein the molar ratio of (R)-methyl oxazaborolidine (MeCBS) 6 to 2-[3-[3-[2-(7-chloro-2-quinolinyl)ethenyl]-phenyl]-3-oxopropyl]benzoic acid methyl ester 2 is at least about 0.01:1.

6. The process of claim 2, wherein the molar ratio of (R)-methyl oxazaborolidine 6 (MeCBS) 6 to 2-[3-[3-[2-(7-chloro-2-quinolinyl)ethenyl]-phenyl]-3-oxopropyl]benzoic acid methyl ester 2 is at least about 0.15:1.

7. The process of claim 3, wherein the organic solvent is tetrahydrofuran (THF), 2-methyl-tetrahydrofuran, diethyl ether, diisopropyl ether, t-butyl methyl ether, dichloromethane, ethyl acetate or a mixture thereof.

8. The process of claim 7, wherein the organic solvent is tetrahydrofuran (THF).

9. The process of claim 3, wherein the reaction is quenched with methanol, ethanol, n-propanol, isopropanol, n-butanol, sec-butanol, isobutanol, or a mixture thereof.

10. The process of claim 9, wherein the reaction is quenched with methanol.

11. The process of claim 1, further comprising acidifying the reaction mixture with an acid.

12. The process of claim 11, wherein the acid is hydrochloric acid, hydrobromic acid, sulfuric acid, p-toluenesulfonic acid, trifluoroacetic acid or a combination thereof.

13. The process of claim 11, wherein the acid is hydrochloric acid.

14. The process of claim 1, comprising adding the 2-[3-[3-[2-(7-chloro-2-quinolinyl)ethenyl]-phenyl]-3-oxopropyl]benzoic acid methyl ester 2 to the catalyst over a period of at least about 30 minutes.

15. The process of claim 1, wherein the reduction is performed at a temperature of about 10° C.

16. A process for purifying methyl 2-[3-(S)-[3-[2-(7-chloro-2-quinolinyl)-ethenyl]phenyl]-3-hydroxypropyl]benzoate 5, the process comprising:
dissolving crude methyl 2-[3-(S)-[3-[2-(7-chloro-2-quinolinyl)-ethenyl]phenyl]-3-hydroxypropyl]benzoate 5 in a polar organic solvent;
adding a non-polar solvent and water;
optionally adding another portion of the non-polar solvent cooling sufficiently to produce crystals of methyl 2-[3-(S)-[3-[2-(7-chloro-2-quinolinyl)-ethenyl]phenyl]-3-hydroxypropyl]benzoate 5;
isolating the crystals by filtration; and
optionally washing and drying the crystals.

17. The process of claim 16, wherein the polar solvent is acetonitrile, acetone, tetrahydrofuran (THF), 2-methyl-tetrahydrofuran, dichloromethane, ethyl acetate or a mixture thereof.

18. The process of claim 17, wherein the polar solvent is tetrahydro-furan (THF).

19. The process of claim 16, wherein the non-polar solvent is pentane, hexane, cyclohexane, heptane, petrol ether or a mixture thereof.

20. The process of claim 19, wherein the non-polar solvent is heptane.

21. The process of claim 16, wherein the non-polar solvent/polar solvent/water ratio in the crystallization step is about 96:40:1 (v/v/v).

22. The process of claim 16, comprising washing the isolated crystals with tetrahydrofuran (THF) and heptane in a ratio of about 1:6 (v/v) tetrahydrofuran (THF):heptane.

23. The process of claim 16, wherein the enantiomeric excess of the isolated crystals is at least about 98%.

24. The process of claim 16, wherein the enantiomeric excess of the isolated crystals is at least about 99.6%.

25. A process for producing methyl 2-[3-(S)-[3-[2-(7-chloro-2-quinolinyl)-ethenyl]phenyl]-3-hydroxypropyl]benzoate 5, the process comprising stereoselectively reducing 2-[3-[3-[2-(7-chloro-2-quinolinyl)ethenyl]-phenyl]-3-oxopropyl]benzoic acid methyl ester 2 in the presence of a catalytic amount of a chiral reduction catalyst comprising (R)-methyl oxazaborolidine (MeCBS) 6, (−)-B-bromodiisopinocamphenylborane, trans-RuH(η¹BH₄)[(R)-2,2′-bis(di-4-tolylphosphino)-1,1′-binaphthyl][(R,R)-1,2-diphenyl-ethylenediamine, trans-RuCl₂ [(R)-2,2′-bis(di-3,5-dimethylphenylphosphino)]-1,1′-binaphthyl][(R,R)-1,2-diphenylethylenediamine], or [[N(S),N′(S), 1R, 2R]-N,N′-bis-[[2-(diphenylphosphino)-phenyl]methyl]1,2-cyclohexanediamine-N,N′,P,P′]-dichloro-ruthenium.
